(19) United States
(12) Patent Application Publication
(10) Pub. No.: US 2025/0186689 A1
(43) Pub. Date: Jun. 12, 2025

(54) NEEDLE-FREE CONNECTORS

(71) Applicant: CareFusion 303, Inc., San Diego, CA (US)

(72) Inventors: Ravikumar Mohan, Chennai (IN); Aman Desai, Bengaluru (IN); Amarsinh Deeliprao Jadhav, Hyderabad (IN)

(21) Appl. No.: 18/530,918

(22) Filed: Dec. 6, 2023

(51) Int. Cl. A61M 5/162; A61M 39/10; A61M 39/26

(52) U.S. Cl. CPC A61M 5/162; A61M 39/10; A61M 39/26

(57) ABSTRACT

Needle-free connectors having a housing with an cavity and center-post, and valves having a head and a skirt are disclosed where the valve is positioned within the cavity of the housing with the center-post extending into a passage through the valve, and the skirt extending away from the head of the valve to the housing to fluidly separate the cavity into a first portion and a second portion, and where an outer end of the skirt is spaced apart from an end of the cavity to permit the valve to move between a closed configuration and an open configuration such that, in the open configuration, the skirt extends along the head and in a direction toward a first housing port, and as the valve moves from the open configuration toward the closed configuration, a volume of the first portion of the cavity decreases to expel any fluid accumulated therein.

### TECHNICAL FIELD

The present disclosure relates generally to medical fluid connectors and, more particularly, to needle-free connectors with a valve configured to resist unintended accumulation of fluid into the needle-free connectors.

### BACKGROUND

Needle-free connectors, including neutral displacement needle-free connectors, offer a solution for providing medical fluid to patients in a safe, efficient, and effective manner. The needle-free connectors permit fluid transmission through the connector, such as between a medical fluid supply and a catheter line without requiring a needle to pierce a connector membrane. In some instances, needle-free connectors comprise a large interior volume that can result in a large amount of residual fluid within the needle-free connector after use of the needle-free connectors. Among other things, the large amount of residual fluid, which was intended to be administered to a patient, is not actually administered to the patient and can result in contamination of the residual fluid and the needle-free connector.

Needle-free connectors can include a valve positioned within a housing such that, upon inserting a medical connector into the needle-free connector, the valve is moved to an open configuration and engagement of the medical connector against the valve forms a fluid path between the medical connector and a fluid pathway of the needle-free connector.

In some instances, a fluid can leak from the fluid pathway at or around the area where the medical connector engages against the valve, resulting in an unintended accumulation of fluid in the needle-free connector. The accumulation of fluid in the needle-free connector can degrade performance of the needle-free connector, including causing failure of the valve of the needle-free connector to compress or return to a neutral position as intended. Additionally, the accumulated fluid may result in incomplete or less than intended fluid flow through the needle-free connector. In some instances, the accumulated fluid may result in the development of biofilms in the needle-free connector which may expose a patient to catheter-associated infection.

### SUMMARY

In accordance with at least some embodiments disclosed herein is the realization that unintended accumulation of fluid in a needle-free connector can degrade performance or cause failure of the needle-free connector. Further, the present disclosure contemplates that an unintended accumulation of fluid in a needle-free connector can be caused by leakage from a fluid pathway formed between the valve of the needle-free connector and a medical connector engaged against the valve. Furthermore, the present disclosure contemplates features that permit observation of the interior of the needle-free connector that can facilitate observation and identification of circumstances that may result in unintended performance of the needle-free connector.

Aspects of the present disclosure provide a needle-free connector comprising a housing having a first housing port, a second housing port, a cavity extending through the first housing port to a cavity floor, and a center-post extending into the cavity and toward the first housing port, the center-post having an opening and an inner surface forming a lumen extending between the opening and the second housing port, and the present disclosure further provides a valve comprising a head and a skirt, the head having an inner surface forming a passage through the head, and the skirt extending radially outward from the head to an outer end of the skirt, the valve disposed around the center-post with the head of the valve longitudinally movable along the center-post and relative to the outer end of the skirt, and the outer end of the skirt spaced apart from the cavity floor to fluidly separate the cavity of the housing into a first portion of the cavity between the skirt and the first housing port, and a second portion of the cavity between the skirt and the second housing port, the valve having a closed configuration wherein the inner surface of the valve is engaged against the opening to resist movement of a fluid through the center-post, and an open configuration wherein the head is biased away from the opening to permit movement of a fluid through the center-post.

In some instances, the present disclosure provides a needle-free connector comprising a housing having a cap portion having a first housing port, a base portion having a second housing port, and a body portion extending from the cap portion to the body portion, wherein a valve seat is formed between the cap portion and the body portion, an inner surface of the housing forms a cavity between the base portion and the cap portion, and a center-post extends into the cavity, the center-post having a proximal end, a distal end, an opening, and an inner surface forming a lumen extending between the opening and the second housing port, wherein the center-post is oriented with the proximal end at the base portion and the distal end between the valve seat and the first housing port, and the present disclosure also provides a valve comprising a head and a skirt, the head having a top end, a bottom end, and an inner surface forming a passage through the head, wherein the center-post extends into the passage, and the skirt extending radially outward from an inner end of the skirt at the head to an outer end of the skirt coupled to the valve seat, wherein the head is movable along the center-post between a closed configuration and an open configuration, such that, in the closed configuration, the bottom end of the head is spaced apart from the proximal end of the center-post by a first distance, and the inner surface of the valve is engaged against the opening of the center-post to resists movement of a fluid through the opening, and in the open configuration, the bottom end of the head is spaced apart from the proximal end of the center-post by a second distance, that is less that the first distance, and the inner surface of the valve is spaced apart from the opening of the center-post to permit movement of a fluid through the opening.

In some instances of the present disclosure provides a needle-free connector comprising a housing having a first housing port, a second housing port, an inner surface forming a cavity extending to the first housing port, and a center-post extending into the cavity and toward the first housing port, the center-post comprising an opening and an inner surface forming a lumen extending between the opening and the second housing port, and the present disclosure provides a valve comprising a head and a skirt, the head disposed around the center-post, and the skirt extending radially outward from the head to an outer end of the skirt, wherein the valve comprises a closed configuration wherein an inner surface of the valve is engaged against the opening of the center-post to resists movement of a fluid therethrough, and an open configuration wherein the head is displaced in a direction away from the first housing port such that the inner surface of the valve is spaced apart from the opening of the center-post to permit movement of a fluid therethrough, and the skirt comprises a skirt length from the head to the outer end of the skirt that increases when the valve moves from the closed configuration toward the open configuration.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV set coupled to a patient, in accordance with aspects of the present disclosure.

FIG. 2 illustrates a cross-sectional view of a needle-free connector, in accordance with aspects of the present disclosure.

FIG. 3 illustrates an exploded cross-sectional view of the needle-free connector of FIG. 2, in accordance with aspects of the present disclosure.

FIG. 4 illustrates a cross-sectional view of the needle-free connector of FIG. 2 with a valve in an open configuration, in accordance with aspects of the present disclosure.

FIG. 5 illustrates a detail view of the needle-free connector of FIG. 4, in accordance with aspects of the present disclosure.

FIG. 6 illustrates a cross-sectional view of the needle-free connector of FIG. 2 with a valve between an open configuration and a closed configuration, in accordance with aspects of the present disclosure.

FIG. 7 illustrates a cross-sectional view of another embodiment of a needle-free connector with a valve in a closed configuration, in accordance with aspects of the present disclosure.

FIG. 8 illustrates a cross-sectional view of the needle-free connector of FIG. 7 with a valve in an open configuration, in accordance with aspects of the present disclosure.

FIG. 9 illustrates a perspective view of a valve for a needle-free connector, in accordance with aspects of the present disclosure.

FIG. 10 illustrates a perspective view of another embodiment of a valve for a needle-free connector, in accordance with aspects of the present disclosure.

FIG. 11 illustrates a perspective view of another embodiment of a valve for a needle-free connector, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present disclosure includes various features and advantages for providing needle-free connectors that can resist accumulation of fluid in an area of the needle-free connector such that the intended performance, efficiency of operation, and safety during use of the needle-free connectors are preserved.

The needle-free connectors of the present disclosure can include a valve positioned within a cavity of a housing and movable relative to the housing. The valve includes a head and a skirt, where the skirt extends away from the head and is coupled to the housing or to another structure of the needle-free connector. The skirt is coupled to the housing or other structure of the needle-free connector to form a seal between the valve and housing, and to define a first portion of the cavity and a second portion of the cavity, where the first and second portions of the cavity are fluidly isolated or separated from each other.

The first portion of the cavity is configured to receive a fluid that may leak into the needle-free connector during use, and the skirt can resist movement of the fluid toward the second portion of the cavity. The first portion of the cavity is formed between any of a first housing port, a head of the valve, a skirt, and/or the housing or other structure of the needle-free connector. The second portion of the cavity is formed between any of the head of the valve, the skirt, a distal end portion of the cavity or cavity floor, and/or the housing. The skirt is coupled to the housing at a location that is spaced apart from the cavity floor such that the skirt is stretched or elongates when the head of the valve is moved in a direction away from the first housing port.

The valve can be moved in a direction away from the first housing port, i.e., from a closed configuration to an open configuration, when a medical connector is inserted into the first housing port to engage and move the head of the valve. As the valve is moved from the closed configuration toward the open configuration, the skirt stretches or is elongated and the volume of the first portion of the cavity increases. Any fluid that is accumulated in the first portion of the cavity, such as fluid that has leaked from between the medical connector and the valve, is retained in the first portion of the cavity to resist movement of the fluid toward the second portion of the cavity.

As the valve moves from the open configuration toward the closed configuration, such as when the medical connector is withdrawn from the needle-free connector, the valve moves toward the first housing port. Movement of the valve toward the first housing port causes the volume of the first portion of the cavity to decrease, relative to the volume of the first portion when the valve is in the open configuration, such that any fluid accumulated in the first portion of the cavity is ejected or displaced out of the needle-free connector.

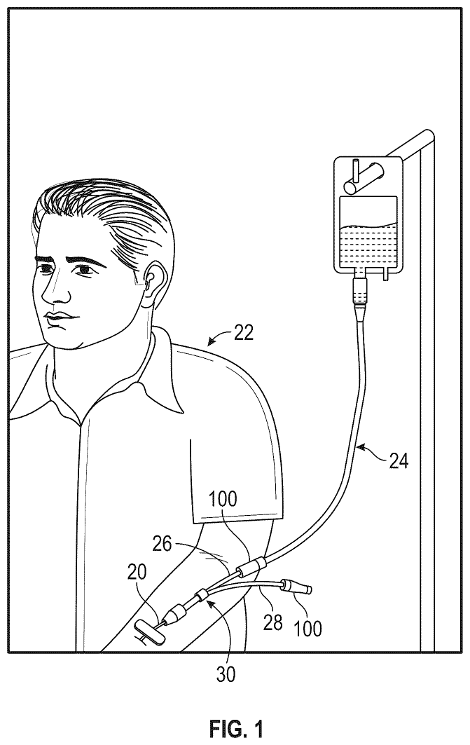

The needle-free connectors of the present disclosure can be used to transmit fluid through a fluid pathway coupled to the needle-free connector. For example, the needle-free connectors can be coupled with a catheter to permit injection of a medicament into the catheter or to withdraw a fluid from the catheter. An example of a needle-free connector 100 coupled with a catheter 20 is illustrated in FIG. 1 with a patient 22 and an intravenous (IV) set 24 coupled to the patient 22. The fluid pathway between the IV set 24 and the catheter 20 includes a y-site 30 and a needle-free connector 100. The y-site 30 includes a first branch 26 and a second branch 28, each comprising a needle-free connector 100 coupled thereto. The IV set 24 is fluidly coupled to the catheter 30 by coupling to the needle-free connector 100 of the first branch 26, while the needle-free connector 100 of the second branch 28 permits injection or withdrawal of a fluid relative with the y-site 30. The needle-free connector 100 is configured with a female luer connector, or similar needle-free connector structure, to permit coupling a medical connector to any of a first housing port or second housing port of the needle free connector. For example, a syringe with a luer connector can be coupled to the needle-free connector 100 to inject a medicament through the needle-free connector 100 or to withdraw a fluid from the needle-free connector 100. In some aspects of the present disclosure, the needle-free connector can be used for high-pressure injection through the needle-free connector.

To permit or resist the movement of fluid through the needle-free connector 100, the needle-free connector includes a valve having a closed configuration and an open configuration. In the closed configuration, the valve resists the movement of fluid through a fluid pathway of the needle-free connector 100, and in the open configuration, the valve permits the movement of fluid through the fluid pathway of the needle-free connector 100. In the example shown in FIG. 1, the needle-free connector 100 coupled to the first branch 26 has a valve in the open configuration, and the needle-free connector 100 coupled to the second branch 28 has a valve in the closed configuration.

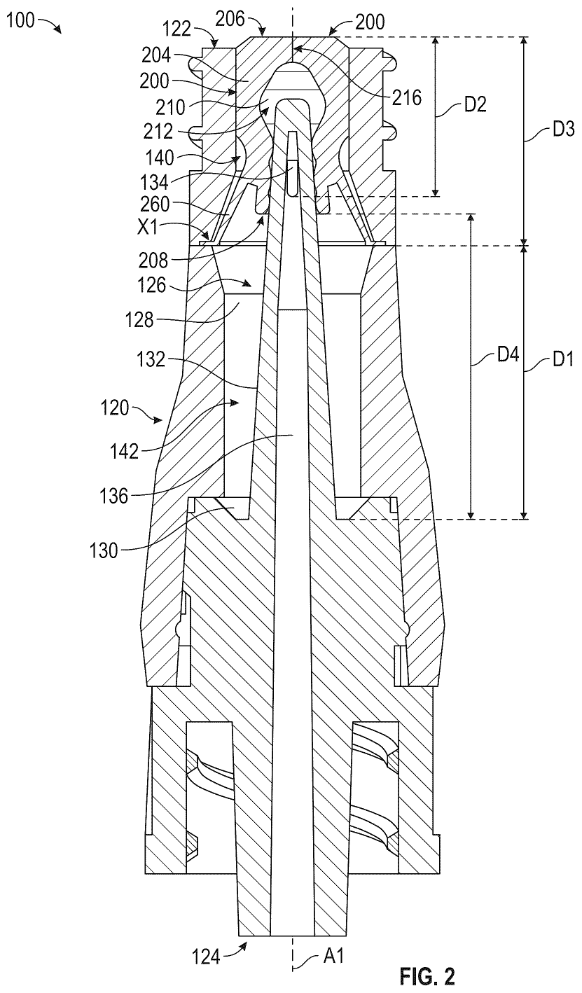

A needle-free connector 100 with a valve in the closed configuration is shown in the cross-sectional view of the needle-free connector 100 in FIG. 2. The needle-free connector 100 includes a housing 120 and a valve 200 that is movable, relative to the housing 120, when the valve 200 moves between the closed configuration and the open configuration.

The housing 120 includes a first housing port 122, a second housing port 124, and a cavity 126. The inner cavity 126 is formed by an inner surface 128 of the housing so that the cavity 126 extends through the first housing port 122 and into the needle-free connector 100. A portion of the cavity 126 is also formed by a cavity floor 130 which can define an end portion of the cavity that is distal to the first housing port 122.

The needle-free connector 100 also includes a center-post 132 that extends into the cavity and in a direction toward the first housing port 122. The center-post 132 includes an opening 134 and a lumen 136 forming a fluid pathway from the second housing port 124 to the opening 134.

The center-post 132 defines a longitudinal axis A1 that extends between the first housing port 122 and the second housing port 124. Thus, a fluid pathway through the needle-free connector 100 extends along a path through the first housing port 122, the opening 134 of the center-post, the lumen 136 of the center-post, and the second housing port 124. a portion of the fluid pathway through the needle-free connector 100 is also formed by the valve 200.

The valve 200 is positioned in the needle-free connector 100 so that the valve is movable, relative to the opening 134 of the center-post, between the closed configuration and the open configuration. The valve 200 includes a head 204 and a skirt 260. The head 204 has a top end 206 and a bottom end 208, where the bottom end is opposite to the top end 206. An inner surface 210 of the head forms a passage 212 that extends through the top end 206 of the head and the bottom end 208 of the head.

The portion of the passage 212 that extends through the top end 206 can be formed, in some embodiments of the present disclosure, by a split septum 214. The split septum 214 is formed by the top end 206 of the valve having a slit 216 that extends through the top end 206 to the passage 212. When the valve 200 is in the closed configuration, the slit 216 is closed and the top end 206 of the valve forms a continuous surface. The continuous surface permits the top end 206 of the valve to be disinfected when the valve 200 is in the closed configuration.

The valve 200 also includes a skirt 260 that extends from the head 204 to the housing 120 to fluidly separate the cavity 126 of the housing into a first portion of the cavity 140 between the skirt 260 and the first housing port 122, and a second portion of the cavity 142 between the skirt 260 and the second housing port 124. In some instances of the present disclosure, the second portion of the cavity 142 is formed between the skirt 260 and the cavity floor 130.

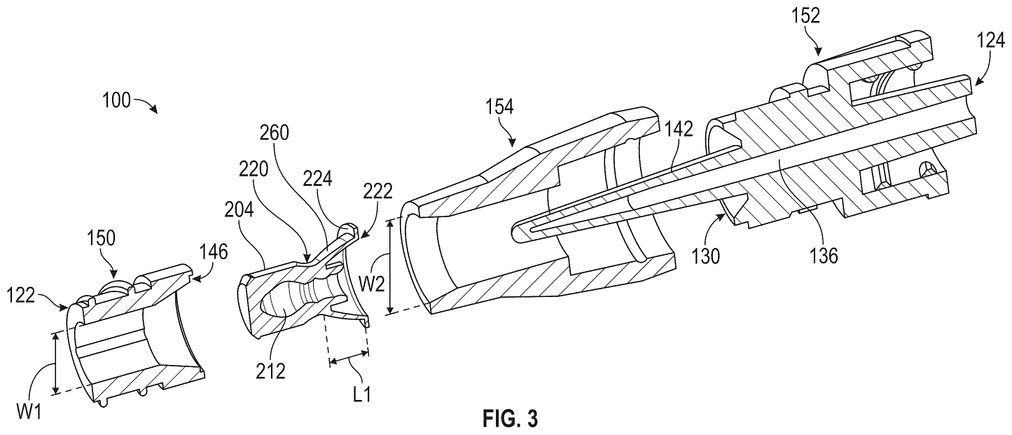

To separate the cavity 126 of the housing into the first portion of the cavity 140 and the second portion of the cavity 142, the skirt 260 includes an inner end 220 couped to the head 204 and an outer end 222 distal to the head 204. The skirt 260 extends radially outward from the head 204 to the outer end 222 of the skirt, as shown in FIGS. 2 and 3.

The outer end 222 of the skirt is spaced apart from the cavity floor 130 to fluidly separate the cavity 126 of the housing into the first portion of the cavity 140 and the second portion of the cavity 142. The outer end 222 of the skirt is engaged against the housing 120 at a location X1 that is between the first housing port 122 and the cavity floor 130. The location X1 where the outer end 222 is engaged against the housing 120 can be formed by a valve seat 146 that is configured to engage against the outer end 222 of the skirt. The valve seat at the location X1 can resist movement of the outer end 222 of the skirt when the head 204 of the valve moves between the closed configuration and the open configuration.

Although the outer end 222 of the skirt is engaged against or coupled to the housing 204, the present disclosure contemplates embodiments in which the skirt 260 is engaged against or coupled to another portion of the needle-free connector 100 to fluidly separate the cavity 126 of the housing into the first portion of the cavity 140 and the second portion of the cavity 142. For example, the outer end 222 of the skirt can engage against or be coupled to a sleeve positioned with in the cavity, a channel of the housing, and/or another structure of the needle-free connector 100. Additionally, the present disclosure contemplates that the skirt 260 can be the same material or unitarily formed with the head 204, or the skirt 260 can be a different material and/or separately formed from the head 204.

In some embodiments of the present disclosure, the outer end 222 of the skirt comprises a lip 224 that is configured to engage against the housing to retain the outer end 222 of the skirt with the housing. In some instances, the lip 224 is positioned in the valve seat and the housing engages against or compresses the lip 224.

The location X1 at which the outer end 222 of the skirt is engaged against the housing 120 is spaced apart from the cavity floor so that the head 204 of the valve can move in a direction away from the first housing port. The location X1 at which the outer end 222 of the skirt is engaged against the housing 120 is also configured so that the head 204 is positioned between the outer end 222 of the skirt and the cavity floor 130 when the valve 200 is in the open configuration. The position of the head 204 and the skirt 260 when the valve is in the open configuration maintains separation between the first portion of the cavity 140 and the second portion of the cavity 142, resists any fluid accumulated in the first portion of the cavity 140 from moving into the second portion of the cavity 142, and causes any fluid accumulated in the first portion of the cavity 140 to be ejected from the needle-free connector 100 when the head moves toward the closed configuration.

The position of the head 204 and the skirt 260 when the valve is in the open configuration can also be configured so that the skirt 200 is stretched or elongated when the valve 200 moves from the closed configuration toward the open configuration, and the skirt 200 returns to an unstretched or neutral configuration to move the head 204 of valve from the open configuration toward the closed configuration.

In some embodiments of the present disclosure, the location X1 at which the outer end 222 of the skirt is engaged against the housing 120 is spaced apart from the cavity floor 130 by a distance D1. The distance D1 is approximately greater than or equal to a distance D2 between the opening 134 of the center-post and the top end 206 of the valve. In some embodiments, the distance D1 is approximately equal to or greater than a distance D3 between the location X1 and the top end 206 of the valve. The relative distances, such as distance D1 being greater than or equal to D2, permits the head 204 to move to a position between the opening 134 and the cavity floor 130, and to permit a fluid flow through the opening 134, when the valve is in the open configuration.

The needle-free connector 100, in some embodiments, can have a housing that is formed by more than one portion that are joined together. For example, referring to FIG. 3, the housing 120 can be formed by a cap portion 150, a base portion 152, and a body portion 154. The cap portion 150 forms the first housing port 122 and a portion of the cavity 126. The base portion 152 forms the second housing port 124 and the center-post 132. The body portion 154 forms another portion of the cavity 126. The cap portion 150, the base portion 152, and the body portion 154 are assembled together so that the body portion 154 extends between the base portion 152 and the cap portion 150, and the center-post 132 extends within the cavity, from the base portion 154 toward the first housing port 122. Additionally, the valve seat 146 is formed by a channel or a ridge that extends into the cap portion 150 so that the lip 222 of the valve is retained between the cap portion 150 and the body portion 154.

The cavity 126 of the housing 120 has a cross-sectional width that the is transverse to the longitudinal axis A1. The cross-sectional width of the cavity can be uniform or can vary between the first housing port 122 and the cavity floor 130. In some embodiments of the present disclosure, the cavity 126 comprises a first width W1 at the first housing port, and a second width W2 at the location X1 at which the outer end 222 of the skirt is engaged against the housing 120, where the second width W2 is greater than the first width W1. When the valve 200 is in the closed configuration, the skirt 260 is oriented extending from the location X1 in a direction that is toward the first housing port 122, and when the valve 200 is in the open configuration, the skirt the skirt 260 is oriented extending from the location X1 in a direction that is away the first housing port 122.

In some embodiments of the present disclosure, the cross-sectional width of the housing 120 tapers in a direction from the location X1 toward the first housing port 122 such that the cross-sectional width of the housing 120 decreases in a direction from the location X1 toward the first housing port 122. In some embodiments of the present disclosure, the cross-sectional width of the housing 120 tapers in a direction of the housing 120 decreases in a direction from the location X1 toward the second housing port 124.

The cross-sectional width of the cavity having the second width W2 at the location X1 being greater than the first width X1 can provide a space for the skirt 260 to transition or change directions when the valve moves between the closed configuration and the open configuration. In some examples of the needle-free connector, the second width W2 permits the skirt 260 to transition from extending in the direction toward the first housing port 122 when the valve is in the closed configuration, to extending in the direction away the first housing port 122 when the valve is in the open configuration.

When the valve 200 is in the closed configuration, the inner surface 210 of the head is engaged against the opening 134 of the center-post to obstruct the lumen of the center-post and resists movement of a fluid therethrough. When the head 204 of the valve is moved in a direction away from the first housing port 122, the valve 200 moves toward the open configuration and the inner surface 210 of the valve becomes spaced apart from the opening 134 of the center-post to permit movement of a fluid therethrough. In some aspects of the present disclosure, when the valve 200 is in the closed configuration, the bottom end 208 of the head is spaced apart from the cavity floor 130 or a proximal end of the center-post by a fourth distance D4, and the fourth distance D4 decreases as the valve is moved from the closed configuration toward the open configuration.

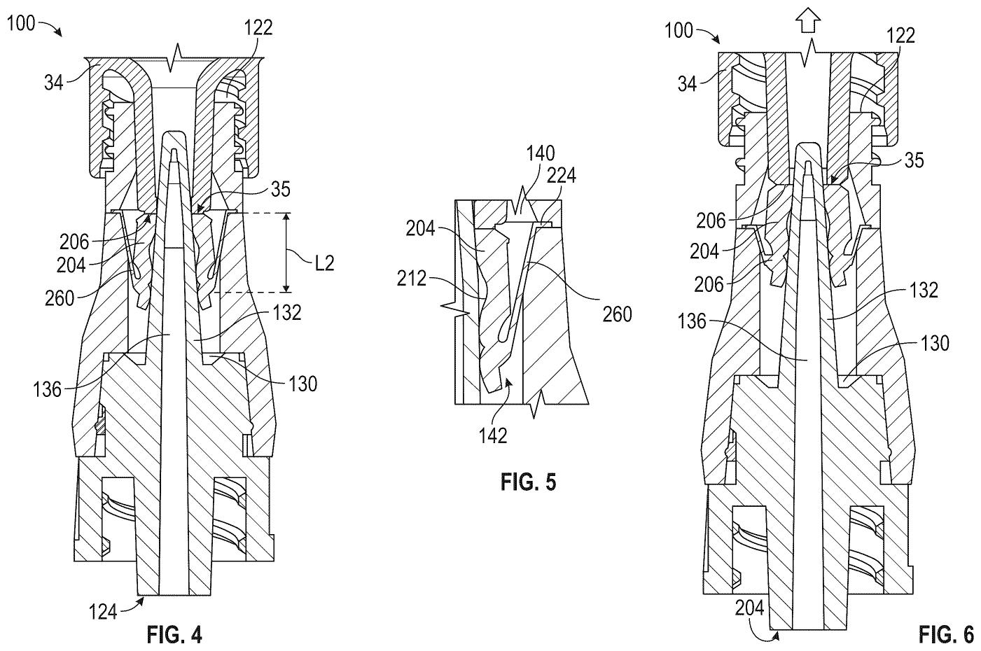

The open configuration of the valve is shown, for example, in FIG. 4, wherein a medical connector 34 having a luer fitting is inserted into the first housing port 122 to displace or move the head 204 of the valve along the center-post 132 and away from the first housing port 122. As the luer fitting of the medical connector 34 is inserted into the first housing port 122, the tip 35 of the luer fitting is engaged against the top end 206 of the valve, and a fluid pathway is formed between the medical connector 34 and the needle-free connector 100. The fluid pathway of the needle-free connector 100 includes the opening 134 and the lumen 136 of the center-post, and the second housing port 124.

To resist movement of fluid out of the fluid pathway, the valve 200 forms a primary seal between the inner surface 210 of the head and an outer surface of the center-post 132, and the skirt 260 forms a secondary seal between the head 204 and the inner surface 128 of the housing. As fluid is directed from the medical connector 34 into the fluid pathway of the needle-free connector 100, or from the fluid pathway of the needle-free connector 100 into the medical connector 34, fluid that may leak out of the fluid pathway, from between the tip 35 of the luer fitting engaged against the top end 206 of the valve, is accumulated or trapped in the first portion of the cavity 140 between the skirt 260 and the first housing port 122, as shown in FIG. 5.

The fluid that is accumulated or trapped in the first portion of the cavity 140 can be ejected out of the needle-free connector 100 when the valve 200 moves from the open configuration toward the closed configuration. The valve 200 can move from the open configuration toward the closed configuration when the medical connector 34 is withdrawn from the needle-free connector 100, as shown in FIG. 6.

Because the second portion of the cavity 142 is fluidly separated from the first portion of the cavity 140, the second portion of the cavity 142 comprises a first volume when the valve 200 is in the closed configuration and a second volume when the valve 200 is in the open configuration, where the second volume is less than the first volume. As the valve 200 moves from the closed configuration toward the open configuration, and the volume of the second portion of the cavity 142 decreases, the pressure within the second portion of the cavity 142 increases. The increases pressure within the second portion of the cavity 142 can cause the valve 200 to move back to the closed configuration when the medical connector 34 is removed or withdrawn from the needle-free connector 100.

Additionally, in some embodiments of the present disclosure, the skirt 260 has a length L1 from the inner end 220 to the outer end 222, when the valve is in the closed configuration. The needle-free connector 100 can be configured so that the skirt 260 has the length L1 when the valve 200 is in the closed configuration or when the valve 200 is in a neural or unbiased state. As the valve 200 moves from the closed configuration toward the open configuration, the skirt 260 is stretched or elongated to a length L2. When the medical connector 34 is removed or withdrawn from the needle-free connector 100, the stretched or elongated skirt 260 can cause the valve 200 to move toward the closed configuration.

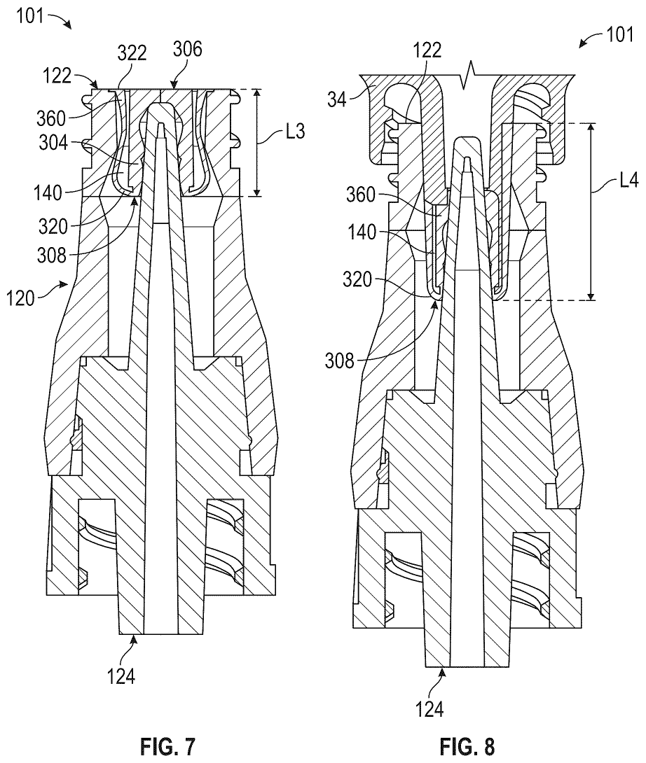

In another embodiment of the present disclosure, the outer end of the skirt can be coupled to a portion of the housing 120 that is between the bottom end of the head and the first housing port 122. Referring to FIGS. 7 and 8, an embodiment of needle-free connector 101 with a valve 300 is illustrated. The valve 300 has a skirt 360 with an inner end 320 of the skirt extending from the head 304 of the valve to an outer end 322 of the skirt at the first housing port 122. The inner end 320 of the skirt extends from the bottom end 308 of the head, and in some embodiments, the inner end 320 of the skirt extends from between the bottom end 308 and the top end 306 of the head. When the valve 300 is in the closed configuration, illustrated in FIG. 7, the top end 306 of the head is aligned with the outer end 322 of the skirt.

In the closed configuration of the valve 300, the top end 306 of the head, the outer end 322 of the skirt, and the portion of the housing forming the first housing port 122 are aligned with a common plane. The alignment of the valve 300 and the housing 120 provide a continuous or unbroken surface that can be easily disinfected and can resist contamination.

When a medical connector 34 is engaged against and moves the head 304 and of the valve in a direction away from the first housing port 122, the valve 300 is moved from the closed configuration toward the open configuration, as illustrated in FIG. 8. As the valve 300 is moved toward the open configuration, the skirt 360 is stretched or elongated from a length L3 to a length L4, wherein length L4 is greater than length L3.

The valve 300 is also configured so that the skirt 360 is positioned between a portion of the medical connector 34 inserted into the first housing port 122 and the inner surface of the housing 120. In some instances of the present disclosure, a portion of the skirt, e.g., the outer end 322, is compressed between the medical connector 34 and the housing 120. Engagement of the medical connector 34 against the skirt 360 can prevent the skirt from becoming displaced when the valve is moved toward the open configuration. When the medical connector 34 is removed or withdrawn from the needle-free connector 101, the stretched or elongated skirt 360 can cause the head 360 to move toward the outer end 322 of the skirt such that the valve 200 moves toward the closed configuration.

As with the valve 200 described in other portions of the present disclosure, fluid that may leak out of the fluid pathway between a medical connector 34 and the top end 306 of the valve, is accumulated or trapped in the first portion of the cavity 140 between the skirt 360 and the first housing port 122. The fluid that is accumulated or trapped in the first portion of the cavity 140 is ejected out of the needle-free connector 101 when the valve 300 moves from the open configuration to the closed configuration. The valve 200 can move from the open configuration to the closed configuration when the medical connector 34 is withdrawn from the needle-free connector 101.

In some embodiments of the present disclosure, the needle-free connector comprises a material that permits observation of an inner portion thereof. For example, the material of the needle-free connector can be configured to permit observation of the center-post and/or the valve. In some embodiments of the present disclosure, the material of the needle-free connector permits observation of the valve being in any of the open configuration and the closed configuration of the valve, and/or fluid moving along the fluid pathway of the needle-free connector. In some aspects of the present disclosure, the material of the needle-free connector, such as the housing and/or the valve can be comprised of a material that is semi-opaque or transparent.

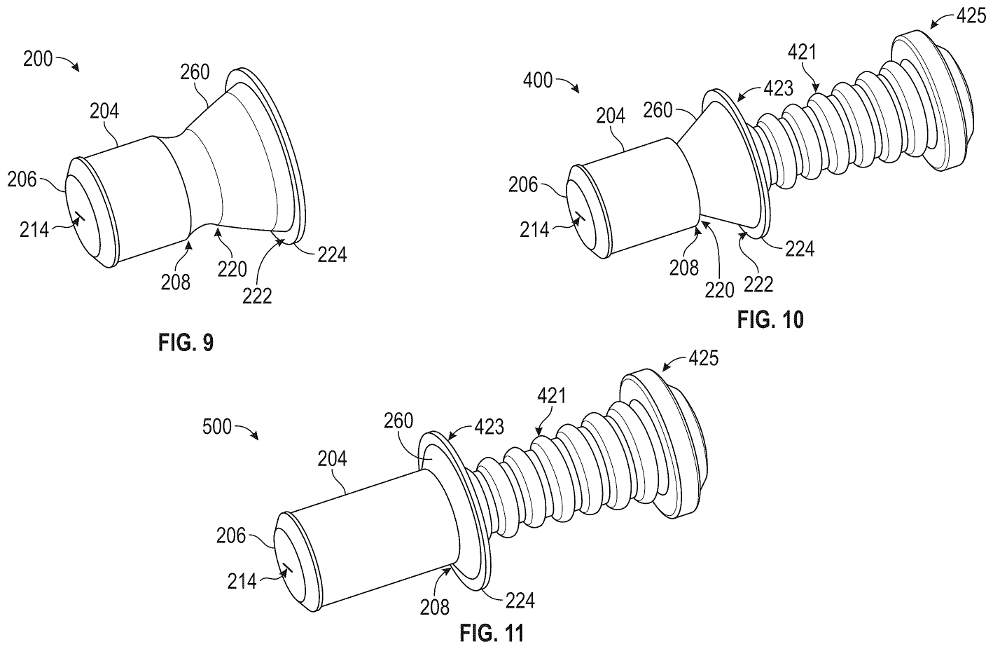

Referring to FIGS. 9-11, several embodiments of the valve for the needle-free connector are illustrated. FIG. 9 illustrates a perspective view of a valve 200 having a head 204 and a skirt 260 that extends from the head. FIGS. 10 and 11 each illustrate a perspective view of an embodiment of a valve 400, 500 having a head 204, a skirt 260 extending from the head 204, and a stem 421. For clarity and brevity, common features between the valve are represented by like reference numbers.

Each of the valves 200, 400, 500 include a head 204 and a skirt 260. The head 204 has a top end 206 and a bottom end 208, where the bottom end is opposite to the top end 206. An inner surface of the head forms a passage that extends through the top end 206 and the bottom end 208 of the head, and a portion of the passage that extends through the top end 206 is formed by a split septum 214.

In some embodiments of the present disclosure, the valve 200 has a skirt 260 with an inner end 220 that is coupled to the bottom end 208 of the head to form an outer surface of the head having a concave surface profile, as shown in at least FIGS. 2, 3, and 9. The concave surface profile of the head 204 extends entirely around the perimeter of the valve 200. When the valve 200 is in the open configuration, the skirt 260 can extend along the concave surface profile to form at least a part of the first portion of the cavity 140 therebetween.

In some embodiments of the present disclosure, the valve 400 has a skirt 260 with an inner end 220 that is coupled to the bottom end 208 of the head at a position that is radially inward relative to the outer surface of the head 204. Thus, a transition from the bottom end 208 of the head to the inner end 220 of the skirt forms an undercut or step. When the valve 400 is in the open configuration, the skirt 260 can extend over and along the undercut to form at least a part of the first portion of the cavity 140 therebetween.

In some embodiments of the present disclosure, the valve 500 has a skirt 260 with an inner end 220 that is coupled to the bottom end 208 of the head such that the skirt extends radially outward from the head 204 at an angle that is transverse relative to the outer surface of the head 204. When the valve 500 is in the open configuration, the skirt 260 can fold over at the transition between the head 204 and the skirt 260 to form at least a part of the first portion of the cavity 140 therebetween.

Additionally, the valve 400, 500 can include a stem 421 having a first end 423 that extends from any of the head 204 and/or the skirt 260, in a direction away from the top of the head end 206 of the head. The stem 421 also has a second end 425 such that the top end 206 of the head forms a distal-most end of the valve and the second end 425 of the stem forms a proximal-most end of the valve.

In embodiments of the valve having a stem 421, the skirt 260 is configured so that the outer end 222 of the skirt is positioned between the top end 206 of the head and the second end 425 of the stem when the valve is in the closed configuration.

When the valve is positioned in the needle-free connector, the stem 421 extends from the head 204 toward the cavity floor 130. The stem 421 has a length L5 between the first end 423 and the second end 425 that is configured so that the second end 425 can engage against the cavity floor 130 or another portion of the needle-free connector. The length of the stem can be approximately the same as the distance between the bottom end of the head and the cavity floor 130. In some instances, the length of the stem is greater than the distance between the bottom end of the head and the cavity floor 130 so that the stem provide a force against the head of the valve in the direction of the first cavity port. In some embodiments, the length of the stem is the same as the distance D4.

When the valve 400, 500 is moved from the closed configuration toward the open configuration, the stem 421 is elastically compressed. When a medical connector 34 is removed or withdrawn from the needle-free connector, the stem 421 expands to direct the head 204 of the valve to move toward the first housing port 122, thereby directing the valve 400, 500 toward the closed configuration. In some embodiments of the present disclosure, the stem 421 is formed as a bellows between the first end 423 and the second end 425.

The features of the present disclosure provide needle-free connectors with a valve having a head and a skirt that are configured to resist unintended accumulation of fluid that can leak into in a needle-free connector by capturing the fluid, resisting movement of the fluid, and expelling the fluid from the needle-free connector. Thus, the features of the present disclosure provide needle-free connectors with improved reliable performance, and which provide for complete transfer of fluid through the needle-free connector, while also resisting the development of contamination in the needle-free connector.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 8 or clause 15. The other clauses can be presented in a similar manner.

Clause 1. A needle-free connector comprising: a housing comprising a first housing port, a second housing port, a cavity extending through the first housing port to a cavity floor, and a center-post extending into the cavity and toward the first housing port, the center-post having an opening and an inner surface forming a lumen extending between the opening and the second housing port; and a valve comprising a head and a skirt, the head having an inner surface forming a passage through the head, and the skirt extending radially outward from the head to an outer end of the skirt, the valve disposed around the center-post with the head of the valve longitudinally movable along the center-post and relative to the outer end of the skirt, and the outer end of the skirt spaced apart from the cavity floor to fluidly separate the cavity of the housing into a first portion of the cavity between the skirt and the first housing port, and a second portion of the cavity between the skirt and the second housing port, the valve having a closed configuration wherein the inner surface of the valve is engaged against the opening to resist movement of a fluid through the center-post, and an open configuration wherein the head is biased away from the opening to permit movement of a fluid through the center-post.

Clause 2. The needle-free connector of Clause 1, wherein the head of the valve comprises a split septum forming a portion of the passage that extends through a top end of the head.

Clause 3. The needle-free connector of any of Clauses 1 and 2, wherein, in the closed configuration, an inner surface of the housing is engaged against the head and a portion of the passage through a top end of the head is obstructed, and in the open configuration, the inner surface of the housing is spaced apart from the head and the center-post extends through the portion of the passage through the top end of the head.

Clause 4. The needle-free connector of any of the preceding Clauses, wherein the outer end of the skirt is positioned between the opening of the center-post and the cavity floor.

Clause 5. The needle-free connector of any of the preceding Clauses, wherein, when the valve moves from the closed configuration toward the open configuration, the head of the valve moves relative to the outer end of the skirt.

Clause 6. The needle-free connector of any of the preceding Clauses, wherein the outer end of the skirt comprises a lip forming an outer perimeter of the skirt.

Clause 7. The needle-free connector of any of the preceding Clauses, wherein the skirt comprises a skirt length from an inner end of the skirt at the head to the outer end of the skirt, and the head comprises a head length from a top end of the head to a bottom end of the head, and wherein the skirt length is less than the head length.

Clause 8. The needle-free connector of any of the preceding Clauses, wherein, in the open configuration, the skirt extends outward from the head in a direction toward a top end of the head.

Clause 9. The needle-free connector of any of the preceding Clauses, wherein, in the closed configuration and the open configuration, the skirt extends outward from the head in a direction toward a top end of the head.

Clause 10. The needle-free connector of any of the preceding Clauses, wherein the skirt comprises a skirt length from an inner end of the skirt at the head to the outer end of the skirt, and the head comprises a head length from a top end of the head to a bottom end of the head, and wherein the skirt length is greater than the head length.

Clause 11. The needle-free connector of any of the preceding Clauses, wherein, in the closed configuration, the first portion of the cavity comprises a first volume, and in the open configuration, the first portion of the cavity comprises a second volume, and wherein the second volume is greater than the first volume.

Clause 12. The needle-free connector of any of the preceding Clauses, wherein, in the closed configuration, the second portion of the cavity comprises a first volume, and in the open configuration, the second portion of the cavity comprises a second volume, and wherein the second volume is greater than the first volume.

Clause 13. The needle-free connector of any of the preceding Clauses, wherein the valve comprises a stem that extends from a bottom end of the head in a direction away from a top end of the head, and wherein the skirt extends around the stem.

Clause 14. The needle-free connector of Clause 13, wherein the stem comprises a first end and a second end, the first end is coupled to the head and the outer end of the skirt is longitudinally positioned between the head and the second end of the stem.

Clause 15. The needle-free connector of Clause 13, wherein the stem is formed as a resiliently compressible bellows between a first end and a second of the stem.

Clause 16. The needle-free connector of any of the preceding Clauses, wherein the opening extends radially from the lumen in a direction that is transverse relative to a longitudinal axis of the center-post.

Clause 17. A needle-free connector comprising: a housing comprising a cap portion having a first housing port, a base portion having a second housing port, and a body portion extending from the cap portion to the body portion, wherein a valve seat is formed between the cap portion and the body portion, an inner surface of the housing forms a cavity between the base portion and the cap portion, and a center-post extends into the cavity, the center-post having a proximal end, a distal end, an opening, and an inner surface forming a lumen extending between the opening and the second housing port, wherein the center-post is oriented with the proximal end at the base portion and the distal end between the valve seat and the first housing port; and a valve comprising a head and a skirt, the head having a top end, a bottom end, and an inner surface forming a passage through the head, wherein the center-post extends into the passage, and the skirt extending radially outward from an inner end of the skirt at the head to an outer end of the skirt coupled to the valve seat; wherein the head is movable along the center-post between a closed configuration and an open configuration, such that, in the closed configuration, the bottom end of the head is spaced apart from the proximal end of the center-post by a first distance, and the inner surface of the valve is engaged against the opening of the center-post to resists movement of a fluid through the opening, and in the open configuration, the bottom end of the head is spaced apart from the proximal end of the center-post by a second distance, that is less that the first distance, and the inner surface of the valve is spaced apart from the opening of the center-post to permit movement of a fluid through the opening.

Clause 18. The needle-free connector of Clause 17, wherein, the valve is positioned with the center-post extending into the passage such that a first distance from the bottom end of the head to the distal end of the center-post is less than a second distance from the bottom end of the head to the proximal end of the center-post.

Clause 19. The needle-free connector of Clause 17, wherein, in the closed configuration, the skirt extends from the valve seat in a direction toward the first housing port, and in the open configuration, the skirt extends from the vale seat in a direction away from the first housing port.

Clause 20. The needle-free connector of Clause 17, wherein the cavity comprises has a first width at the first housing port and a second width at the valve seat, and wherein the second width is greater than the first width.

Clause 21. The needle-free connector of Clause 17, wherein the cavity comprises has a width that decreases in a direction from the valve seat toward the first housing port and in a direction from the valve seat away from the first housing port.

Clause 22. A needle-free connector comprising: a housing comprising a first housing port, a second housing port, an inner surface forming a cavity extending to the first housing port, and a center-post extending into the cavity and toward the first housing port, the center-post comprising an opening and an inner surface forming a lumen extending between the opening and the second housing port; and a valve comprising a head and a skirt, the head disposed around the center-post, and the skirt extending radially outward from the head to an outer end of the skirt, wherein the valve comprises a closed configuration wherein an inner surface of the valve is engaged against the opening of the center-post to resists movement of a fluid therethrough, and an open configuration wherein the head is displaced in a direction away from the first housing port such that the inner surface of the valve is spaced apart from the opening of the center-post to permit movement of a fluid therethrough, and the skirt comprises a skirt length from the head to the outer end of the skirt that increases when the valve moves from the closed configuration toward the open configuration.

Clause 23. The needle-free connector of Clause 22, wherein, when the valve moves from the closed configuration toward the open configuration, the head of the valve moves relative to the outer end of the skirt.

Clause 24. The needle-free connector of Clause 22, wherein, in the closed configuration, the skirt extends from the head in a direction away from a top end of the head, and in the open configuration, the skirt extends from the head in a direction toward a top end of the head.

Clause 25. The needle-free connector of any of Clauses 22 to 24, wherein the valve comprises a stem that extends around the center-post and the skirt extends around the stem.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A needle-free connector comprising:
a housing comprising a first housing port, a second housing port, a cavity extending through the first housing port to a cavity floor, and a center-post extending into the cavity and toward the first housing port, the center-post having an opening and an inner surface forming a lumen extending between the opening and the second housing port; and
a valve comprising a head and a skirt, the head having an inner surface forming a passage through the head, and the skirt extending radially outward from the head to an outer end of the skirt, the valve disposed around the center-post with the head of the valve longitudinally movable along the center-post and relative to the outer end of the skirt, and the outer end of the skirt spaced apart from the cavity floor to fluidly separate the cavity of the housing into a first portion of the cavity between the skirt and the first housing port, and a second portion of the cavity between the skirt and the second housing port, the valve having a closed configuration wherein the inner surface of the valve is engaged against the opening to resist movement of a fluid through the center-post, and an open configuration wherein the head is biased away from the opening to permit movement of a fluid through the center-post.

2. The needle-free connector of claim 1, wherein the head of the valve comprises a split septum forming a portion of the passage that extends through a top end of the head.

3. The needle-free connector of claim 1, wherein, in the closed configuration, an inner surface of the housing is engaged against the head and a portion of the passage through a top end of the head is obstructed, and in the open configuration, the inner surface of the housing is spaced apart from the head and the center-post extends through the portion of the passage through the top end of the head.

4. The needle-free connector of claim 1, wherein the outer end of the skirt is positioned between the opening of the center-post and the cavity floor.

5. The needle-free connector of claim 1, wherein, when the valve moves from the closed configuration toward the open configuration, the head of the valve moves relative to the outer end of the skirt.

6. The needle-free connector of claim 1, wherein, in the closed configuration, the skirt extends outward from the head in a direction away from a top end of the head, and in the open configuration, the skirt extends outward from the head in a direction toward a top end of the head.

7. The needle-free connector of claim 1, wherein, in the closed configuration and the open configuration, the skirt extends outward from the head in a direction toward a top end of the head.

8. The needle-free connector of claim 1, wherein, in the closed configuration, the second portion of the cavity comprises a first volume, and in the open configuration, the second portion of the cavity comprises a second volume, and wherein the second volume is greater than the first volume.

9. The needle-free connector of claim 1, wherein the valve comprises a stem that extends from a bottom end of the head in a direction away from a top end of the head, and wherein the skirt extends around the stem.

10. The needle-free connector of claim 9, wherein the stem comprises a first end and a second end, the first end is coupled to the head and the outer end of the skirt is longitudinally positioned between the head and the second end of the stem.

11. The needle-free connector of claim 9, wherein the stem is formed as a resiliently compressible bellows between a first end and a second of the stem.

12. A needle-free connector comprising:
a housing comprising a cap portion having a first housing port, a base portion having a second housing port, and a body portion extending from the cap portion to the body portion, wherein a valve seat is formed between the cap portion and the body portion, an inner surface of the housing forms a cavity between the base portion and the cap portion, and a center-post extends into the cavity, the center-post having a proximal end, a distal end, an opening, and an inner surface forming a lumen extending between the opening and the second housing port, wherein the center-post is oriented with the proximal end at the base portion and the distal end between the valve seat and the first housing port; and
a valve comprising a head and a skirt, the head having a top end, a bottom end, and an inner surface forming a passage through the head, wherein the center-post extends into the passage, and the skirt extending radially outward from an inner end of the skirt at the head to an outer end of the skirt coupled to the valve seat;
wherein the head is movable along the center-post between a closed configuration and an open configuration, such that, in the closed configuration, the bottom end of the head is spaced apart from the proximal end of the center-post by a first distance, and the inner surface of the valve is engaged against the opening of the center-post to resists movement of a fluid through the opening, and in the open configuration, the bottom end of the head is spaced apart from the proximal end of the center-post by a second distance, that is less that the first distance, and the inner surface of the valve is spaced apart from the opening of the center-post to permit movement of a fluid through the opening.

13. The needle-free connector of claim 12, wherein, the valve is positioned with the center-post extending into the passage such that a first distance from the bottom end of the head to the distal end of the center-post is less than a second distance from the bottom end of the head to the proximal end of the center-post.

14. The needle-free connector of claim 12, wherein, in the closed configuration, the skirt extends from the valve seat in a direction toward the first housing port, and in the open configuration, the skirt extends from the vale seat in a direction away from the first housing port.

15. The needle-free connector of claim 12, wherein the cavity comprises has a first width at the first housing port and a second width at the valve seat, and wherein the second width is greater than the first width.

16. The needle-free connector of claim 12, wherein the cavity comprises has a width that decreases in a direction from the valve seat toward the first housing port and in a direction from the valve seat away from the first housing port.

17. A needle-free connector comprising:
a housing comprising a first housing port, a second housing port, an inner surface forming a cavity extending to the first housing port, and a center-post extending into the cavity and toward the first housing port, the center-post comprising an opening and an inner surface forming a lumen extending between the opening and the second housing port; and
a valve comprising a head and a skirt, the head disposed around the center-post, and the skirt extending radially outward from the head to an outer end of the skirt, wherein the valve comprises a closed configuration wherein an inner surface of the valve is engaged against the opening of the center-post to resists movement of a fluid therethrough, and an open configuration wherein the head is displaced in a direction away from the first housing port such that the inner surface of the valve is spaced apart from the opening of the center-post to permit movement of a fluid therethrough, and the skirt comprises a skirt length from the head to the outer end of the skirt that increases when the valve moves from the closed configuration toward the open configuration.

18. The needle-free connector of claim 17, wherein, when the valve moves from the closed configuration toward the open configuration, the head of the valve moves relative to the outer end of the skirt.

19. The needle-free connector of claim 17, wherein, in the closed configuration, the skirt extends from the head in a direction away from a top end of the head, and in the open configuration, the skirt extends from the head in a direction toward a top end of the head.

20. The needle-free connector of claim 17, wherein the valve comprises a stem that extends around the center-post and the skirt extends around the stem.
